(19) United States
(12) Patent Application Publication
(10) Pub. No.: US 2006/0116637 A1
(43) Pub. Date: Jun. 1, 2006

(54) Tract measuring device having a unitary occluded tip and inflatable sock member and method of making the same

(75) Inventors: Nathan Shirley, Herriman, UT (US); Donald J. McMichael, South Jordan, UT (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/000,211

(22) Filed: Nov. 30, 2004

(51) Int. Cl. A61M 29/00

(52) U.S. Cl. 604/104; 264/500; 604/96.01; 604/910

(57) ABSTRACT

A stoma or tract measuring device and method of using the same. A tract measuring device of the present invention may generally include a shaft having a first and a second end; and an inflatable sock molded in situ at one end of the shaft; the inflatable sock having a distal end and a proximal end, the inflatable sock having an occluded tip with a predetermined geometry integrally formed at the distal end of the sock and an open proximal end; wherein the open end of the sock is secured to the shaft; and wherein the shaft has a lumen in communication with the inflatable sock so as to allow for inflation and deflation of the sock.

The present invention generally relates to medical devices, and more specifically medical devices used for obtaining measurements.

Catheterization of a body cavity is frequently performed in medical procedures either to insert substances into or to remove substances from the body. During many of these procedures, it is necessary to keep the catheter in a relatively stable position to perform the desired insertion or removal. With the use of enteral feeding catheters (i.e., catheters which enable the administration of nutritional solutions directly into the stomach or intestines), for example, it is necessary to ensure that the catheter is not accidentally removed from the stomach or intestines. This is true both during the actual administration or removal of fluids, and the time periods in between.

In order to ensure that a catheter is maintained in the proper position, it is common to use a balloon disposed near the distal (patient) end of the catheter shaft. Inflating the balloon causes the balloon to contact the anatomical structure (i.e., a duct or stomach wall) and thereby prevents the catheter from moving out of the proper position. In the case of enteral feeding, a stoma is formed leading into the stomach or intestine. The catheter is positioned to extend through the stoma so as to form a channel into the stomach or intestines through which enteral feeding solutions may be instilled.

FIG. 1 shows a side view of a prior art balloon catheter 10 having a head 14 disposed at a proximal end 15 of the catheter 10. The head 14 contains valves (not shown) which regulate the flow of fluids through the balloon catheter 10. The head 14 also prevents the balloon catheter 10 from completely advancing through the stoma and into the stomach or intestine of the user.

To prevent the catheter 10 from being pulled out of the stomach/intestinal wall, a balloon 18 is disposed along a catheter shaft 26. The catheter 10 is shown having an optional stiff tip 30, which is attached to the catheter shaft 26 at a distal end 17 opposite the head 14. The catheter shaft 26 is typically made of a medical grade silicone. The stiff tip 30, when present, is also frequently formed of a medical grade silicone but is usually configured to be at least as rigid as the catheter shaft 26.

The balloon 18 has a balloon proximal end 20 attached to the catheter shaft 26 by the use of adhesive, thereby forming a proximal cuff 32. Likewise, the balloon distal end 22 is adhesively attached to the catheter shaft 26 and/or stiff tip 30, thereby forming a distal cuff 34.

The balloon 18 is advantageous because it allows the catheter shaft 26 to be inserted into the stoma (not shown) while the balloon 18 is uninflated. Once the catheter shaft 26 is properly positioned in the stoma, a syringe (not shown) is inserted into a side port 36 of the head 14 and a fluid is injected into the balloon 18 through a lumen (not shown in FIG. 1) of the catheter 10 so as to inflate the balloon 18.

While the balloon 18 remains inflated, the catheter 10 stays properly positioned in the stoma. The position of the balloon catheter 10 is maintained in such a manner until removal is desired. If the catheter 10 needs to be removed, the balloon 18 may be deflated so that it will not interfere with withdrawal of the catheter shaft 26 and stiff tip 30.

The type of balloon 18 shown in FIG. 1 is fashioned around the perimeter of the catheter shaft 26 such that when it is deflated it reduces or contracts about the shaft 26 but is still clearly larger than overall diameter of the catheter.

Attachment of the balloon 18 to the catheter shaft 26 is frequently accomplished by gluing the balloon proximal end 20 and the balloon distal end 22 to corresponding positions on the external surface of the catheter shaft 26 so as to form a proximal cuff 32 and a distal cuff 34, respectively. Such cuffs 32 and 34 are longitudinal sections of the balloon 18 whose inside diameters correspond to the outside diameter of the shaft 26 at their respective points of attachment to the catheter 10 and have a distance between them which is about the length of the uninflated balloon 18. The cuffs 32 and 34 must be of sufficient length to provide a tight and durable seal between the balloon 18 and the catheter shaft 26.

FIG. 2 shows a side view of another prior art balloon catheter 110. The catheter 110 is generally similar to catheter 10 (FIG. 1) except that the head 114 (FIG. 2) of catheter 110 is a large or non-low profile head and is adapted to extend well beyond the patient's body. While the balloon 18 of catheter 10 may be located at or near the distal end 17 of catheter shaft 26, as shown in FIG. 1, FIG. 2 also shows that balloon 118 may be located more inwardly of the distal end 117 of the catheter 110 (i.e. more proximal to the head 114).

While the prior art balloon configurations shown in FIGS. 1 and 2 work to maintain the balloon catheters 10 and 110, respectively, in the proper position within the patient, those balloon catheters as well as the other known balloon catheters do have disadvantages, especially involving placement. For example, sizing a catheter is important to minimize the trauma to a patient. If a catheter is too small it may cause undue pressure to be exerted on or unnecessarily constrict the patient's tissue. If a catheter is too big, slippage may occur, and the repeated sliding of the catheter along the stoma or tract may lead to irritation and/or infection. The sizing issues are especially significant with low profile enteral feeding devices as the low profile devices are generally not adjustable for different stoma or tract lengths.

Additional difficulties with prior measuring devices are commonly encountered with manufacture of the measuring devices as well as with the placement or insertion of the measuring devices. The prior devices either lacked a tip adapted to assist with placement or the tip was not integrally formed with the device. That is, prior devices were manufactured such that a balloon or sleeve was attached to the shaft of a device in one step and, if present, a tip was attached in another step. Such additional manufacturing steps are undesirable. Further, where present, the tips of prior devices were generally formed by placing a fixed amount of silicone onto the distal end of the catheter shaft and allowing it to gravity form a bulbous tip. The shape of such gravity formed bulbous tips are inconsistent in shape due to forming conditions such as consistency of the silicone, temperature and other conditions of the environment the formation occurs in, and the like.

Further some of the bulbous tips created on the prior devices make insertion of the measuring device harder as the bulbous portion of the tip widens out beyond the width of the catheter shaft thus requiring a tract or stoma width which is larger than necessary for the shaft. As stoma size is generally desired to be as small as possible to achieve the desired supplementing or the like, measurement tools or devices which require either that the stoma width be unnecessarily wide or a stretching of the stoma during insertion would be readily replaced upon the availability of a suitable alternative. Furthermore, a stoma measuring device having a separately formed sleeve and tip also provides for the existence of a distal cuff or exposed bond surface. Such an exposed bond point presents but one more edge or surface to catch on the patient during insertion or placement and cause irritation thereto.

Accordingly, there is a need and desire for a stoma or tract measuring device having a sock member with an integrally formed occluded tip of a predetermined geometry at the distal end thereof.

### SUMMARY OF THE INVENTION

In response to the difficulties and problems discussed above, an improved tract measuring device has been developed.

One aspect of the present invention is tract measuring device which may be used to determine the length of a stoma or other tract opening within a patient. The device may generally include a head or handle, a shaft extending from the head, and an inflatable sock. The head has at least one opening. The shaft having a lumen disposed in communication with the opening. The sock is formed in situ at the distal end of the shaft and is integrally formed with a tip. The tip has a predetermined geometry and is closed or occluded at the distal end of the sock while the proximal end of the sock is open, but is secured to the shaft. The sock is positioned about the shaft such that an expandable cavity is defined between the sock and the shaft and is in fluid communication with the lumen in the shaft.

The measuring device may further include scale indicia along at least a portion of the shaft. Another aspect of the present invention may include a positioning member, having a distal side and proximal side. The measuring device of the present invention may further include a valve to regulate fluid (e.g., gas, liquid, gels, etc.) flow into or out of the lumen and/or expandable cavity and defined between the inflatable sock and the shaft to thereby control inflation or deflation of the expandable cavity. Such a valve will desirably be located in the opening of the head.

The present invention is also directed to a method of making a tract measuring device with an integrally formed inflatable sock and occluded tip. The method generally includes the steps of: providing a shaft having a proximal end and a distal end, the shaft being occluded at the distal end; providing a mold for forming the integral inflatable sock and occluded tip, the mold being such that a resulting sock formed therein will have an open end; positioning the distal end of the shaft within the mold; providing a resin; supplying the resin into the mold; allowing the resin to solidify, harden, cure, set up, or otherwise adapt the shape of the mold and form the integral inflatable sock and tip at the distal end of the shaft; removing the shaft and the integrally formed sock and tip from the mold; and securing the open end of the sock to the shaft. The method may further include the steps of providing a valve, and inserting the valve into a lumen in the shaft so as to allow regulation of fluid flow into and out of a cavity defined between the shaft and the sock. One or more aspects of the present invention may also include the step of providing a slidable positioning member about the shaft.

The present invention is also directed to a method of providing a system of using a tract measuring device, the method generally including the following steps: providing a medical device such as one or more of those discussed in more detail herein, and providing directions for positioning the medical device relative to the patient so as to allow for a determination of the length of the tract, thereby enabling a user of the device to accurately determine the length of the tract the measuring device is positioned within. The method of providing a system of using a tract measuring device of the present invention may further include the step of providing directions to select a feeding device having an appropriate shaft length based on the determined tract length.

The invention will be more fully understood and further features and advantages will become apparent when reference is made to the following detailed description of exemplary aspects of the invention and the accompanying drawings.

### BRIEF DESCRIPTION OF THE DRAWINGS

The purpose and advantages of the present invention will be apparent to those skilled in the art from the following detailed description in conjunction with the appended drawings in which:

FIG. 1 is a view of a prior enteral feeding device;

FIG. 2 is a view of another prior enteral feeding device;

FIG. 3 is a side view of an aspect of a measuring device according to the present invention;

FIG. 4 is a cross-sectional view of the device of FIG. 3;

FIG. 5 is an perspective view of an aspect of the measuring device according to the present invention wherein the measuring device is shown positioned within a patient;

FIG. 6 is a cross-section view of an exemplary mold for forming an integral sock and tip according to the present invention; and

FIG. 7 is a cross-section view of an alternate mold for forming an integral sock and tip according to the present invention.

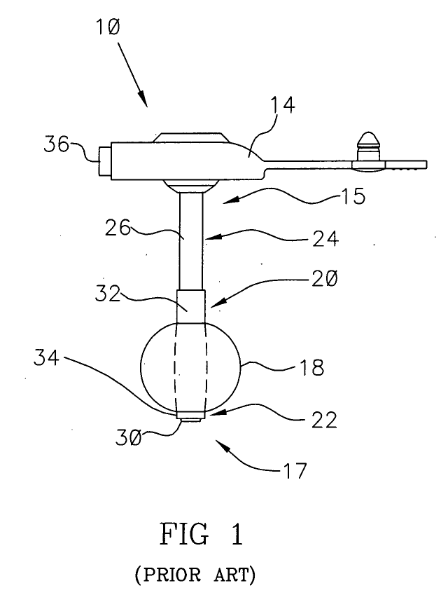

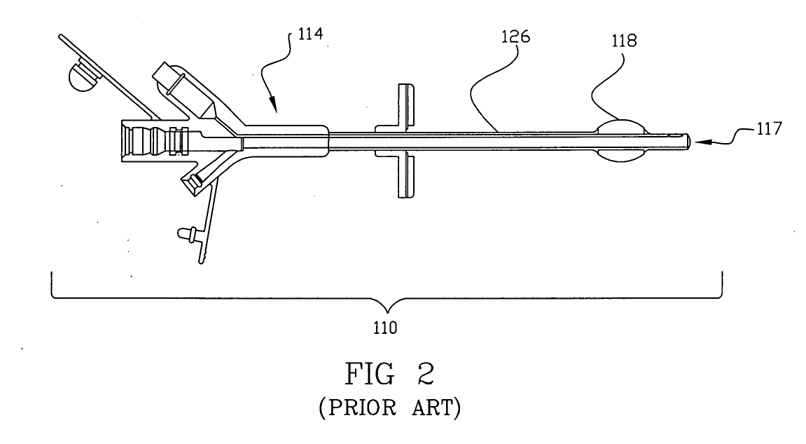

### DETAILED DESCRIPTION OF THE PRESENT INVENTION

Reference now will be made in detail to various embodiments of the invention, one or more examples of which are set forth below. Various elements of the present invention will be given numeral designations and the invention will be discussed so as to enable one skilled in the art to make and use the invention. It should be appreciated that each example is provided by way of explaining the invention, and not as a limitation of the invention. For example, features illustrated or described with respect to one aspect may be used with another aspect to yield still a further aspect. These and other modifications and variations are contemplated to be within the scope and spirit of the invention.

In addition, the invention will be described in the context of its various configurations. It should be appreciated that alternative arrangements of the invention can comprise any combination of such configurations. As such, the use of a desired aspect for ease in understanding and describing the invention shall not, in any manner, limit the scope of the invention.

As used herein, the term “distal” generally refers to the direction of the patient or the end of a device intended to be closest to or inserted the farthest into a patient and the term “proximal” generally refers to the direction of the clinician or the end of a device intended to be furthest from or inserted the least into a patient.

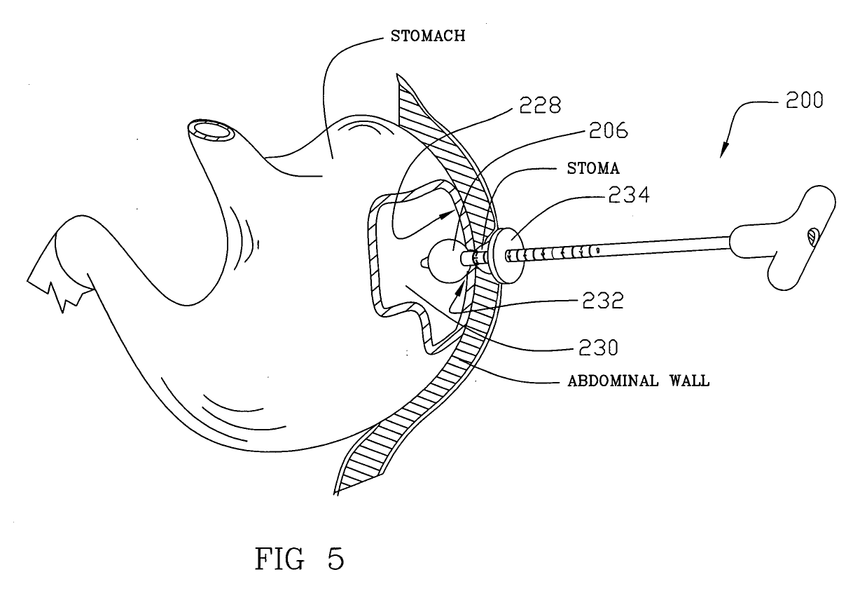

As used herein the term “stoma or tract measuring device” generally refers to a device intended to be introduced into an opening in a body and to allow for measurement of the length or depth of the opening or tract. Although the opening in a body typically begins at an exterior surface and extends to an internal body cavity or duct, as shown at, for example, FIG. 5; other possible configurations and uses are contemplated.

Although the term “sock” is generally used throughout, it is also intended to include, but is not limited to, other suitable inflatable or expandable members with a closed end and an open end.

It will be appreciated that as used herein the terms “inflate”, “expand”, “deploy” or deviations thereof are intended to overlap in meaning and be used interchangeably as they relate to an inflatable sock. Similarly, the terms “deflate”, “contract”, “collapse” or deviations thereof are intended to overlap in meaning and be used interchangeably as they relate to an inflatable sock.

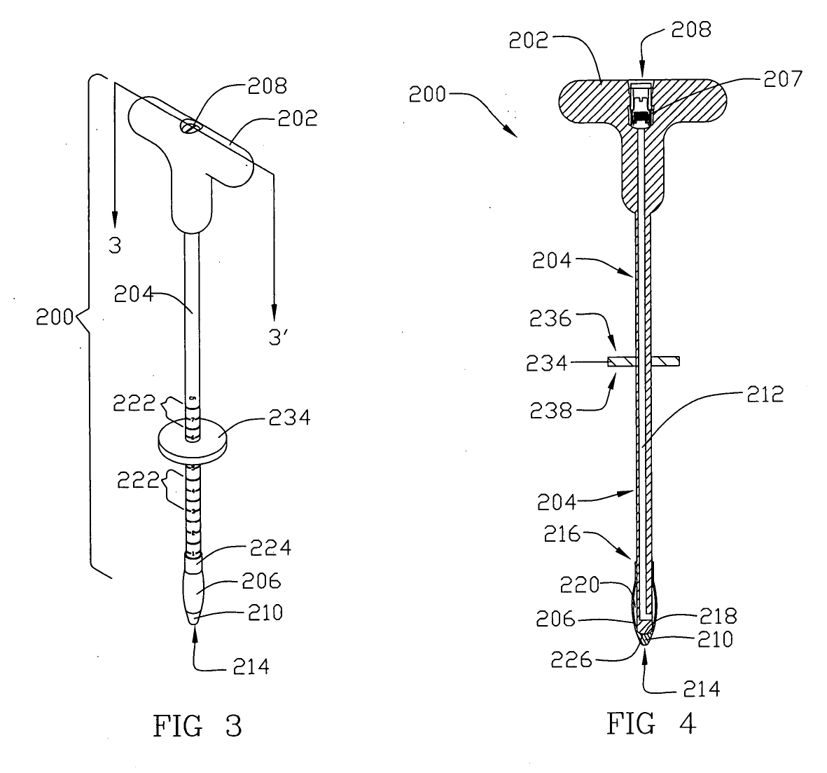

FIG. 3 illustrates a tract measuring device 200 according to the present invention. The tract measuring device may be used to determine the length of a particular stoma or tract in a patient. Such a device is especially useful in determining the proper size of an enteral feeding device to be placed in the patient through the stoma. As illustrated, the measuring device 200 includes a head or handle 202, a shaft 204 extending from the head 202, and an inflatable sock 206. The head 202 has at least one opening 208. The shaft 204 is shown in FIG. 4 having a lumen 212 disposed in fluid communication with the opening 208. The sock 206 is formed in situ at the distal end 218 of the shaft 204 and is integrally formed with a tip 210. The tip 210 which has a predetermined geometry is closed or occluded at the distal end 214 of the sock while the proximal end 216 of the sock 206 is open. In FIGS. 3 and 4, the sock 206 is shown with the proximal end 216 attached to the shaft 204. The sock 206 is positioned about the shaft 204 such that an expandable cavity 220 (FIG. 4) is defined between the sock 206 and the shaft 204 and is in fluid communication with the lumen 212 in the shaft 204.

Desirably the tip 210 of the present invention is tapered so as to provide for easier insertion into or through a stoma or tract in a patient. Although not shown in the figures, in one or more aspects of the present invention, the proximal or open end 216 of the sock 206 may be secured in a recess along the shaft 204 so as to reduce the cuff edge and/or allow for a smooth sock to shaft transition. It will be appreciated that the recess may not be deep enough to receive the entire cuff; however, any reduction in cuff edge exposure is believed to be beneficial.

The measuring device may further include scale indicia such as that shown as 222 along at least a portion of the shaft 204. The scale indicia may take any suitable form or color. It will be appreciated that all suitable sizes and scales of markings or indicia are contemplated. At least one aspect of the present invention contemplates measurement markings every 0.25 cm for a total of 6.0 cm. It is contemplated that the markings may be created in or on the shaft 204 of the measuring device 200 in any suitable manner.

Exemplary suitable manners of creating the markings include the printing of the markers, the molding of the shaft about an insert containing the markings, or the like. In one or more aspects of the present invention the indicia may be selected or printed such that it is visible in low light conditions. Portions of the shaft tubing and/or the indicia may be radiopaque in some aspects of the present invention.

Another aspect of the present invention may include a positioning member, such as slidable disc 234 having a proximal side 236 and distal side 238 as illustrated in FIG. 4.

It will be appreciated by those having skill in the art that the markings 222 will desirably be registered with or otherwise indicative of the distance from the proximalmost point at which the sock contacts the inner surface 228 (FIG. 5) of an inner body cavity 230 (FIG. 5) within the patient to which the tract extends when the measuring device 200 is properly positioned within the patient and the expandable cavity 220 defined by the sock 206 and shaft 202 is expanded or deployed (e.g., when the expandable cavity 220 is fully expanded or deployed or when the expandable cavity 220 may be less than fully expanded or deployed yet provides sufficient resistance to displacement). A discussion of the desired positioning of the measuring device at the time of measurement is described in more detail below. However, for purposes of understanding the proximalmost point at which the sock 206 contacts or rests against the inner surface 228 of the inner body cavity 230 within the patient to which the tract to be measured extends when the measuring device is properly positioned within the patient is exemplarily illustrated at point 232 in FIG. 5. In other embodiments indicia might be registered with the distal end of the shaft 202 or tip 210, or there might be multiple sets of indicia indicating distances from multiple points along the shaft 202, inflatable sock 206, or tip 210.

The head or handle 202 of the measuring device of the present invention may be attached to the proximal end of the shaft 204 in any suitable manner. Exemplary ways of attaching the head 202 to the shaft 204 include but are not limited to adhesive securement or overmolding.

The measuring device 200 of the present invention may further include a valve 207 (FIG. 4) to regulate fluid flow into or out of the lumen 212 and/or expandable cavity 220 and thereby control expansion or deployment of expandable cavity. Such a valve 207 will desirably be located in the opening 208 of the head 202. Any suitable valve is contemplated. An exemplary valve may be a luer lock inflation valve such as that found in the MIC-KEY* low profile gastrostomy feeding tube (available from Ballard Medical Products, a wholly owned subsidiary of the assignee of the present invention). It will be appreciated that while fluid or the like is actually provided into the space or expandable cavity defined between the shaft 204 and the inflatable sock 206, it will be appreciated that references herein to inflation or expansion of the sock or the expandable cavity defined between the sock and the shaft of the device are intended to be interchangeable.

Although the sock 206 is shown in FIGS. 3-4 as being attached to the shaft 204 such that proximal cuff 224 is formed about the shaft and generally extends away from 10 the expandable cavity 220 defined between the cuff 224 and tip 210, it is contemplated that the sock end 216 may be attached to the shaft 204 in an inverted or folded under fashion such that the resulting cuff or point of attachment extends inward relative to the expandable cavity 220. Such an inverted attachment is described in more detail in the context of enteral feeding catheters in U.S. patent application Ser. No. 10/307,057, which is assigned to the assignee of the present invention, and which is incorporated in its entirety herein for all purposes.

As suggested above, the tip 210 should abut the distal end 218 of the shaft 204. That is the proximal end 226 of the tip 210 should abut or contact the distal end 218 of the shaft 204. Desirably, the tip 210 is secured to the distal end 218 of the shaft 204 so as to prevent displacement of the tip 210 relative to the end of the shaft 204. It will be appreciated that the proximal end 226 of the tip 210 may, but need not, be flat. Rather it may and should be shaped to generally correspond to the shape of the distal end 218 of the shaft 204 as suggested in FIG. 4.

It will be appreciated that the sock or the like, etc. may be formed by any acceptable process, including for example, injection molding, dipping, compression molding, extrusion, or the like. Furthermore, it is contemplated that the sock 206 may be secured to the shaft 204 of the measuring device 200 in any suitable manner, including, for example, by adhesive and overmolding.

It will also be appreciated that the sock may be formed so as to allow for controlled expansion or deployment in a particular direction or limit expansion in another. Alternately, a sock may be designed to assume suitable shapes other than the traditional rounded shape. The sock may be designed so as upon expansion it forms such exemplary shapes as tire shaped, apple shaped, oblong, or the like. It will be appreciated that the ability of a stoma or tract measuring device to include an expandable member (e.g., an inflatable sock) which is sized, configured, and attached to the shaft in the same or similar fashion (at least at the proximal end of expandable member) to that which is included on the catheter to be placed within the patient will allow a more accurate sizing in some instances. That is, for example, if the enteral feeding catheter to be placed has a sock or balloon attached or shaped differently (at least at the proximal end thereof) than that on the tract measuring device, it is possible that upon placement of the enteral feeding catheter that the catheter could fit the patient tighter or looser than desired. In some instances the deviation between the shapes or manner of attachment of the sock or balloon on the shaft of the measuring device and enteral feeding catheter may provide negligible or inconsequential differences in the measurements taken, although in some instances the resulting measurements may vary significantly. It will be appreciated that the greater the deviation the more likely a significant difference in measurements is to occur.

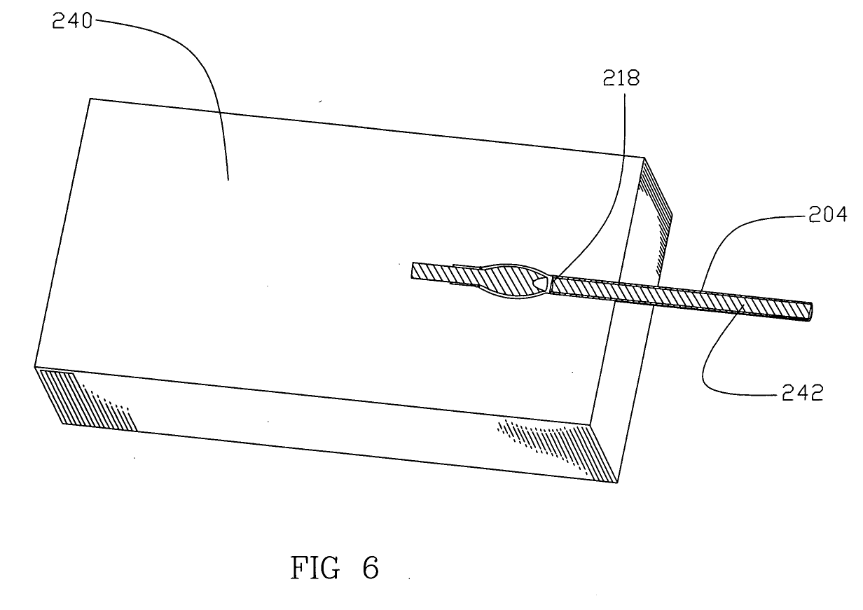

Having generally described an aspect of a tract measuring device of the present invention, the disclosure herein now shifts to a method of making a tract measuring device with an integrally formed inflatable sock and occluded tip. The method generally includes the steps of: providing a shaft 204 having a proximal end and a distal end 218, the shaft being occluded at the distal end; providing a mold 240 (FIG. 6) for forming the integral inflatable sock 206 and occluded tip 210, the mold being such that a resulting sock formed therein will have an open end 216; positioning the distal end 218 of the shaft 204 within the mold 240; providing a resin; supplying the resin into the mold; allowing the resin to solidify, harden, cure, set up or otherwise adapt the shape of the mold and form the integral inflatable sock and tip at the distal end 218 of the shaft 204; removing the shaft 204 and the integrally formed inflatable sock 206 and tip 210 from the mold 240; and securing the open end 216 of the sock 206 to the shaft 204 to define an expandable cavity 220. The method may further include the steps of providing a valve 207, and inserting the valve into a lumen 212 (including the opening in the head or handle 202) in the shaft 204 so as to allow regulation of fluid flow into and out of a cavity 220 defined between the shaft 204 and the inflatable sock 206. One or more aspects of the present invention may also include the step of providing a slidable positioning member 234 about the shaft.

It will be appreciated that while the present invention calls for the distal end of the shaft to be occluded or blocked when it is inserted into the mold, any suitable manner of achieving such an occlusion is contemplated. Exemplary ways of providing for such blockage or occlusion of the distal end of the tube include providing a tube which is manufactured or modified to have a closed end, or inserting a core pin into the shaft so as to block or occlude the distal end of the shaft. Accordingly at least one aspect of the present invention contemplates that a method of the present invention may further include the steps of providing a core pin 242 and positioning the core pin 242 within the shaft 204 so as to occlude the distal end 218 of the shaft 204 (as illustrated in FIG. 6).

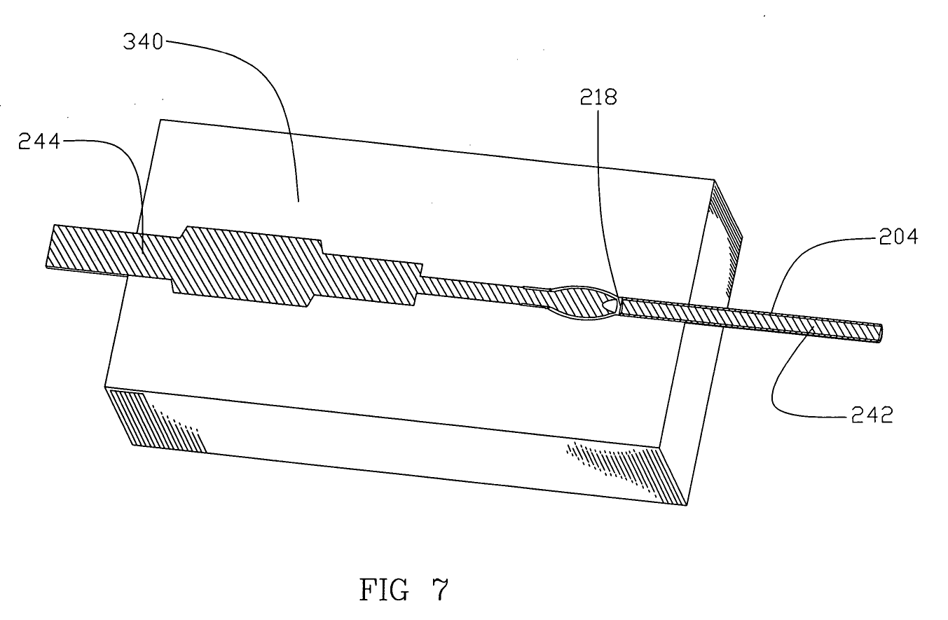

Another aspect of a method of making a tract measuring device in accordance with the present invention contemplates the steps of providing a core pin 244 (FIG. 7) and positioning the core pin 244 within the mold 340 (FIG. 7) so that the integral sock 206 and tip 210 are formed about the core pin 244 within the mold 340. It will be appreciated that the provision of a core pin 244 within the mold 340 so that the integral sock 206 and tip 210 may be formed between the core pin 244 and the mold 340 is completely independent of and may occur with or without the provision of a core pin 242 (FIGS. 6 and 7) within the shaft 204 of the measuring device 200 so as to occlude the distal end 218 of the shaft 204.

The present invention is also directed to a method of providing a system of using a tract measuring device, the method generally including the following steps: providing a medical device such as one or more of those discussed in more detail herein, and providing directions for positioning the medical device relative to the patient so as to allow for a determination of the length of the tract, thereby enabling a user of the device to accurately determine the length of the tract the measuring device is positioned within. The method of providing a system of using a tract measuring device of the present invention may further include the step of providing directions to select a feeding device having an appropriate shaft length based on the determined tract length. That is, the method may provide for utilizing the determined length of the stoma or tract during a medical procedure to select or identify an appropriately sized instrument for enteral feeding. The method could further or alternatively include the step of providing directions for using the length of the tract as measured with the device to select from medical devices having an appropriate length based on the shaft length and the inflatable sock attached to the shaft.

It will be appreciated that the use of the measuring device may generally include inserting the distal end of the measuring device into the tract in the patient; expanding the sock of the device; positioning the measuring device such that the proximal edge of the inflated, expanded, or otherwise deployed sock rests against an inner surface of an inner body cavity within the patient; and determining the distance between the proximal edge of the expanded or deployed sock and the outer surface of the patient's abdominal wall. It will be appreciated that the inner surface of an inner body cavity within the patient upon which the proximal edge of the expanded sock desirably rests is desirably adjacent the distal opening of the tract or stoma being measured.

As discussed above, such a measuring device may further include scale indicia along at least a portion of the shaft. Accordingly, with those aspects of the present invention including the scale indicia, the step of determining the distance between the proximal edge of the expanded sock and the outer surface of the patient's abdominal wall may include utilizing the scale indicia. In those aspects having a positioning member, such as slidable disc 234, having a proximal side 236 and distal side 238, the determination of the size of a tract within a patient may further include moving the positioning bar 234 along the shaft 204 of the device until the distal edge 238 of the positioning member 234 rests against the patient's abdominal wall 228 (FIG. 5) when the measuring device 200 is properly positioned within the patient, and reading or otherwise taking a measurement with the assistance of the positioning member. Of course it will be appreciated that the measurement should be taken once the tract measuring device is properly positioned.

While the invention has been described in detail with respect to specific aspects thereof, those skilled in the art, upon obtaining an understanding of the invention, may readily conceive of alterations to, variations of, and equivalents to the described aspects and the processes for making them. The invention may be embodied in other specific forms without departing from the scope and spirit of the inventive characteristics thereof. The present aspects therefore are to be considered in all respects as illustrative and not restrictive, the scope of the invention being indicated by the appended claims rather than by the foregoing description, and all changes which come within the meaning and range of equivalency of the claims are therefore intended to be embraced therein.

We claim:

1. A medical device comprising:
a shaft having a first and a second end; and
an inflatable sock formed in situ at one end of the shaft;
the inflatable sock having a distal end and a proximal end, the inflatable sock having an occluded tip with a predetermined geometry integrally formed at the distal end of the sock and an open proximal end;
wherein the open end of the sock is secured to the shaft; and
wherein the shaft has a lumen in communication with the inflatable sock so as to allow for inflation and deflation of an expandable cavity defined between the shaft and the inflatable sock.

2. The medical device of claim 1 wherein the tip is tapered.

3. The medical device of claim 1 wherein the tip has a proximal end and the proximal end of the tip abuts the second end of the shaft.

4. The medical device of claim 1 wherein the open end of the sock is secured in a recess along the shaft.

5. The medical device of claim 1 further comprising at least one set of scaled indicia along at least a portion of the shaft.

6. The medical device of claim 1 further comprising a valve to regulate fluid flow into or out of the expandable cavity defined between the shaft and the inflatable sock.

7. The medical device of claim 1 further comprising a positioning member slidably mounted about the shaft of the device.

8. The medical device of claim 1 wherein the shaft has radiopaque markers included therein.

9. A method of making a tract measuring device with an integrally formed inflatable sock and occluded tip, the method comprising the steps of:
providing a shaft having a proximal end and a distal end, the shaft being occluded at the distal end;
providing a mold for forming the integral inflatable sock and occluded tip with a predetermined geometry, the mold being such that the resulting sock formed therein has an open end;
positioning the distal end of the shaft within the mold;
providing a resin;
supplying the resin into the mold;
allowing the resin to solidify and form the integral inflatable sock and tip at the distal end of the shaft;
removing the shaft and the integrally formed sock and tip from the mold; and
securing the open end of the sock to the shaft to define an expandable cavity between the shaft and the inflatable sock.

10. The method of claim 9 further comprising the steps of providing a valve, and inserting the valve into a lumen in the shaft so as to allow regulation of fluid flow into and out of a cavity defined between the shaft and the inflatable sock.

11. The method of claim 9 further comprising the step of providing a slidable positioning member about the shaft.

12. The method of claim 9, wherein the distal end of the shaft is occluded by a core pin or where the distal end of the shaft is blocked.

13. The method of claim 9 further comprising the steps of providing a core pin; and positioning the core pin within the shaft so as to occlude the distal end of the shaft.

14. The method of claim 9 further comprising the steps of providing a core pin; and positioning the core pin within the mold so that the integral sock and tip are formed between the core pin and the mold.

15. A method of providing a system of using a tract measuring device, the method comprising the following steps:
providing a medical device comprising the device of claim 1; and
providing directions for positioning the medical device relative to the patient so as to allow for a determination of the length of the tract;
thereby enabling a clinician to accurately determine the length of the tract the measuring device is positioned within.

16. The method of claim 15, further comprising the step of providing directions to select a feeding device having an appropriate shaft length based on the determined tract length.

17. The method of claim 15 further comprising the step of providing directions for using the length of the tract as measured with the device to select from medical devices having an appropriate length based on the shaft length and the inflatable sock attached to the shaft.
